(19) United States
(12) Patent Application Publication
(10) Pub. No.: US 2024/0424146 A1
(43) Pub. Date: Dec. 26, 2024

(54) DRUG SCREENING METHOD AND MODEL FOR MONITORING PLATINUM-RESISTANT OVARIAN CANCER TUMOR GROWTH AND METASTASES

(71) Applicant: THE BOARD OF REGENTS OF THE UNIVERSITY OF OKLAHOMA, Norman, OK (US)

(72) Inventors: Bethany N. HANNAFON, Edmond, OK (US); Samrita DOGRA, Oklahoma City, OK (US)

(73) Assignee: THE BOARD OF REGENTS OF THE UNIVERSITY OF OKLAHOMA, Norman, OK (US)

(21) Appl. No.: 18/742,139

(22) Filed: Jun. 13, 2024

Related U.S. Application Data
(60) Provisional application No. 63/510,162, filed on Jun. 26, 2023.

(51) Int. Cl. A61K 49/00

(52) U.S. Cl. CPC A61K 49/0008

(57) ABSTRACT

A method of screening a chemical substance as a drug candidate against High-grade serous ovarian carcinoma (HGSOC). Mice with xenograft tumors caused by a cell line comprising HGSOC cells which have been transduced with firefly luciferase gene (Luc), or by a cell line comprising cisplatin-resistant HGSOC cells which have been transduced with Luc are treated with the chemical substance to the mice for a predetermined treatment period. Effects that the chemical substance has on the mice are assessed after the predetermined treatment period. The cell lines may be, for example, OVCAR-8-Luc cells or cisplatin-resistant OVCAR-8-Luc cells.

### STATEMENT REGARDING FEDERALLY SPONSORED RESEARCH OR DEVELOPMENT

Not Applicable.

### CROSS REFERENCE TO RELATED APPLICATIONS

This application claims the benefit of U.S. Provisional Patent Application Ser. No. 63/510,162, filed Jun. 26, 2023, the content of which is hereby expressly incorporated herein by reference in its entirety.

### REFERENCE TO A SEQUENCE LISTING

This application contains a Sequence Listing XML, which has been submitted electronically and is hereby incorporated by reference in its entirety. Said Sequence Listing XML, created on Jun. 13, 2024, is named OKLAP0021US_ST26.xml and is 1,846 bytes in size.

### BACKGROUND

High-grade serous ovarian carcinoma (HGSOC) is the most common malignant type of ovarian cancer and contributes a disproportionate share of OC-associated mortalities. First-line treatments for HGSOC include surgical debulking and platinum-taxol-based chemotherapy drugs.¹ More than 75% of the initial responders relapse with more severe disease due to the emergence of multi-drug resistance and widespread intraperitoneal metastases.^{1,2} HGSOC tumor spread is facilitated by single cells or multi-cellular clusters or “spheroids” that detach from the primary tumor site (ovary, fallopian tube), travel through the peritoneal fluid (ascites), and attach to the peritoneal and omental tissue to form metastases. Methods to model ovarian cancer in vivo are challenged by the ability to monitor tumor growth in real-time in a physiologically relevant model system. The most relevant model system for ovarian cancer growth is the intrabursal and intraperitoneal implantation methods, which are both challenged by the inability of researchers to quantitatively measure tumor growth dynamics. An improved model system for assessing ovarian cancer growth would be of great benefit.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows dose response curves of OVCAR-8 Luc and OVCAR-8 CPR Luc cells. Cells were treated with increasing doses of cisplatin and cell viability was measured using the MTS assay. IC50 values were calculated using non-linear regression analysis in GraphPad Prism software.

FIG. 2 shows luminescent signals from OVCAR-8 CPR Luc cells. Cells were plated in adherent 2D conditions, or as suspended spheroids in 3D conditions. The cells were imaged on a Caliper Life Sciences IVIS Spectrum System.

FIG. 3 shows a schematic of step-wise procedures in the development and evaluation of an in vivo intraperitoneal model of HGSOC.

FIG. 4 shows mouse model timelines and treatment groups in the experiments of the disclosure.

FIG. 5 shows in vivo animal imaging of intraperitoneal OVCAR-8 CPR luc xenograft model. Three mice from each treatment group were imaged weekly beginning at 1 week post OVCAR-8 CPR Luc cell injection. Mice were anesthetized and injected with D-luciferin substrate and imaged on the Caliper Life Sciences IVIS Spectrum System.

FIG. 6A shows tumor weights of mice after treatment with a DCLK1 inhibitor (DCLK1-IN-1), in combination with cisplatin-treatment in the OVCAR-8 CPR luc xenograft model. Mice were administered vehicle control, 25 mg/kg DCLK1-IN-1, 5 mg/kg cisplatin alone or in combination, every other day for 28 days. Tumor weights were determined at necropsy. DCLK1-IN-1 is also known as DCLK inhibitor-1, and has CAS Registry No. 2222635-15-4.

FIG. 6B shows numbers of visible metastases in mice of the experiment of FIG. 6A. Numbers of visible metastases were determined at necropsy.

FIG. 6C shows body weights of mice in the experiment of FIG. 6A. Body weight measurements were obtained weekly during treatment.

FIG. 7A shows tumor weights of mice after treatment with a DCLK1 inhibitor, DCLK1 SAMiRNA, in combination with cisplatin-treatment in the OVCAR-8 CPR luc xenograft model. Mice were administered 30 mg/kg non-targeting SAMiRNA control (siNC), 30 mg/kg SAMiRNA targeted to DCLK1 (siDCLK1), or 5 mg/kg cisplatin alone or in combination, every other day for 28 days. Tumor weights number were determined at necropsy. siDCLK1 has the RNA sequence 5′-GGCGACUUGCCUGAGCGGG-3′ (SEQ ID NO:1).

FIG. 7B shows numbers of visible metastases in mice of the experiment of FIG. 7A. Numbers of visible metastases were determined at necropsy.

FIG. 7C shows body weights of mice in the experiment of FIG. 7A. Body weight measurements were obtained weekly during treatment.

### DETAILED DESCRIPTION

As noted above, conventional model systems for monitoring and analyzing ovarian cancer growth suffer from disadvantages, particularly in regard to quantitative measurement of ovarian tumor growth dynamics. The novel cell lines described herein overcome those shortcomings by enabling the visualization and dynamic monitoring of ovarian tumor growth. Additionally, these cell lines represent the clinical spectrum of ovarian cancer including platinum-chemotherapy sensitive and platinum-chemotherapy resistant cancers. The present disclosure therefore is directed to cell lines and methods of their use in modeling human high-grade serous ovarian carcinoma that, in at least one embodiment, is resistant to platinum-based chemotherapy in vitro and in vivo. The inclusion of the expression of the luciferase gene into the cell lines enables the monitoring and visualization of tumor cells in vitro and in vivo. This model system enables the real-time monitoring of tumor growth and can be used as an assay of the efficacy of therapeutic drugs in inhibiting tumor growth and peritoneal metastases. A pharmaceutical combination therapy aimed at inhibiting DCLK1 activity and platinum-based chemotherapy for the treatment of ovarian cancer is also described.

Before further describing various embodiments of the compositions, cell lines, and methods of the present disclosure in more detail by way of exemplary description, examples, and results, it is to be understood that the compositions, cell lines, and methods of the present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. Thus, while the compositions, cell lines, and methods of the present disclosure have been described in terms of particular embodiments, it will be apparent to those of skill in the art that variations may be applied to the compositions, cell lines, and methods and in the steps or in the sequence of steps of the methods described herein without departing from the concept, spirit, and scope of the inventive concepts.

All patents, published patent applications, and non-patent publications mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). An amino acid sequence having a length in a range of 12 to 50 amino acids, for example, refers to a peptide or oligopeptide oligonucleotide having at least 12 amino acids and less than 51 amino acids, and includes any range bounded by two different integers in said range of 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids, including for example 18 to 25, 20 to 24, or 20-22.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±25%, or ±20%, or ±15%, ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

Where used herein the term “active agent” refers to a presently disclosed or described oligonucleotide, or a compound, complex, coacervate, and/or conjugate comprising the oligonucleotide. By “biologically active” is meant the ability of an active agent to modify the molecular, biochemical, or physiological system of a cell, organ, or organism, without reference to how the active agent has its physiological effects.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Where used herein, the pronoun “we” is intended to refer to all persons involved in a particular aspect of the investigation disclosed herein and as such may include non-inventor laboratory assistants and non-inventor collaborators working under the supervision of the inventor(s).

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. The term “topical” is used herein to define a mode of administration through an epithelial surface, such as but not limited to, the skin, eye, or internal epithelial surfaces. In addition, the compositions of the present disclosure may be designed to provide delayed, controlled, extended, and/or sustained release using formulation techniques which are well known in the art.

The terms “therapeutic composition” and “pharmaceutical composition” refer to a composition containing a peptide as described herein that may be administered to a subject by any method known in the art or otherwise contemplated herein, wherein administration of the composition brings about a therapeutic effect as described elsewhere herein.

The term “effective amount” refers to an amount of a peptide or peptide compound which is sufficient to exhibit a detectable therapeutic, amelioration, or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or a symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

The term “mutant” or “variant” is intended to refer to a protein, peptide, nucleic acid or organism which has at least one amino acid or nucleotide which is different from the wild type version of the protein, peptide, nucleic acid, or organism and includes, but is not limited to, point substitutions, multiple contiguous or non-contiguous substitutions, chimeras, or fusion proteins, and the nucleic acids which encode them.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul (Proc. Natl. Acad. Sci. USA (1990) 87:2264-2268), modified as in Karlin & Altschul (Proc. Natl. Acad. Sci. USA (1993) 90:5873-5877).

In one embodiment “% identity” represents the number of amino acids or nucleotides which are identical at corresponding positions in two sequences of a protein having the same activity or encoding similar proteins. For example, two amino acid sequences each having 100 residues will have 95% identity when 95 of the amino acids at corresponding positions are the same.

Another example of a mathematical algorithm used for comparison of sequences is the algorithm of Myers & Miller (CABIOS (1988) 4:11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman (Proc. Natl. Acad. Sci. USA (1988) 85:2444-2448).

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, Methods in Enzymology (1996) 266:460-480; Altschul et al., J Molec Biol. (1990) 215:403-410; Gish & States, Nature Genetics (1993) 3:266-272; Karlin & Altschul, Proc. Natl. Acad. Sci. USA (1993) 90:5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Specific amino acids may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

The terms “oligonucleotide,” “polynucleotide,” or “nucleic acid,” as used herein, include any nucleotide sequence which encodes a variant, chimeric, or mutant peptide including polynucleotides in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. The polynucleotide sequence encoding a mutant peptide or encoding a therapeutically-effective fragment of a mutant peptide can be substantially the same as the coding sequence of the endogenous coding sequence as long as it encodes a biologically active mutant peptide. Further, the mutant peptide, or therapeutically-effective fragment of a mutant peptide may be expressed using polynucleotide sequence(s) which differ in codon usage due to the degeneracies of the genetic code or allelic variations.

As noted above, the peptides of the present disclosure, and the nucleic acids which encode them, include peptide and nucleic acid variants which comprise additional conservative substitutions. For example, the variant peptides include, but are not limited to, variants that are not exactly the same as the sequences disclosed herein, but which have, in addition to the substitutions explicitly described for various sequences listed herein, conservative substitutions of amino acid residues which do substantially not impair the agonistic or antagonistic activity or properties of the variants described herein. Examples of such conservative amino acid substitutions include, but are not limited to, ala to gly, ser, or thr; arg to gln, his, or lys; asn to asp, gln, his, lys, ser, or thr; asp to asn or glu; cys to ser; gln to arg, asn, glu, his, lys, or met; glu to asp, gln, or lys; gly to pro or ala; his to arg, asn, gln, or tyr; ile to leu, met, or val; leu to ile, met, phe, or val; lys to arg, asn, gln, or glu; met to gln, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

The present constructs or antigen-binding portions thereof can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

In one aspect, the pharmaceutical formulations comprising compositions or nucleic acids, antibodies or fragments thereof are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the polypeptides or nucleic acids have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the compositions are prepared with carriers that will protect the construct or fragment thereof against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The constructs and fragments thereof in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the compositions described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The constructs can be delivered or administered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

Formulated compositions comprising the constructs can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

As used herein, the term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi inhibits the gene by compromising the function of a target RNA, completely or partially. Both plants and animals mediate RNAi by the RNA-induced silencing complex (RISC); a sequence-specific, multicomponent nuclease that destroys messenger RNAs homologous to the silencing trigger. RISC is known to contain short RNAs (e.g., approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

As used herein, the term “siRNA” refers to a short interfering RNA. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand”; the strand homologous to the target RNA molecule is the “sense strand”, and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

In at least certain embodiments, the active agents (e.g., oligonucleotides) may have strand lengths comprising, for example, approximately 12 to 50, or 18 to 40, or 20 to 30 nucleotides, including a targeting sequence (i.e., a seed sequence) that is complementary to a target sequence of a nucleic acid which comprises a portion of an AR coregulator, such as an AR coregulator as listed elsewhere herein, a pre-mRNA transcribed from an AR coregulator, and/or (2) a mature mRNA processed from said pre-mRNA. For example, when an oligonucleotide binds to the target sequence of a preprocessed mRNA, it effectively inhibits splicing at the normal splice acceptor site and thus produces a splice variant mRNA, leading to truncated or otherwise aberrant versions of the encoded protein upon translation, or when the oligonucleotide binds to the target region of a mature mRNA, it effectively inhibits proper translation of the mRNA into an encoded protein.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases as described below. The term “nucleic acid” encompasses the terms “oligonucleotide” and “polynucleotide,” each as a subgenus of the term “nucleic acid.” The term “oligonucleotide” generally refers to a molecule of between about 3 and about 100 nucleobases in length. The term “polynucleotide” generally refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule that comprises a complementary strand or “complement” of a particular sequence comprising a molecule. As used herein, a single-stranded nucleic acid may be denoted by the prefix “ss,” and a double-stranded nucleic acid by the prefix “ds. The terms “polynucleotide sequence” or “nucleic acid,” as used herein, include any polynucleotide sequence which encodes a peptide or fusion protein (or polypeptide) including polynucleotides in the form of RNA, such as mRNA, or in the form of DNA, including, for instance, cDNA and genomic DNA obtained by cloning or produced by chemical synthetic techniques or by a combination thereof. The RNA or DNA may be double-stranded or single-stranded. Single-stranded DNA may be the coding strand, also known as the sense strand, or it may be the non-coding strand, also referred to as the anti-sense strand.

Where a strand is designated herein as RNA, and thus comprises uracil (U) nucleobases, the present disclosure is also directed to an equivalent DNA sequence where the U nucleobase is replaced with a thymine (T) nucleobase. For example, where an RNA active agent described herein comprises the seed sequence GUCUGA, the equivalent DNA active agent comprises the seed sequence GTCTGA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Therefore, in the context of the present disclosure, the term “oligonucleotide” refers to an oligomer or polymer of RNA or DNA or mimetics thereof. This term includes oligonucleotides composed of naturally-occurring nucleobases, sugars and covalent internucleoside (backbone) linkages as well as oligonucleotides having non-naturally-occurring nucleobases, sugars and synthetic heterocycles and covalent internucleoside (backbone) linkages which function similarly. Such modified or substituted non-natural oligonucleotides, as compared to native (natural) forms may have desirable properties such as, for example, enhanced cellular uptake, enhanced affinity for nucleic acid target and increased stability in the presence of nucleases.

Where used herein, the term “oligonucleotide,” is also intended to include linked nucleobase sequences containing modified backbones comprising non-natural internucleoside linkages. As defined in this specification, oligonucleotides having modified backbones include those that retain a phosphorus atom in the backbone and those that do not have a phosphorus atom in the backbone. Further, for the purposes of this specification, the term “nucleoside” is intended to refer to a nucleobase linked to a ribose or deoxyribose sugar (a natural nucleoside), and to a nucleobase linked to a non-ribose or non-deoxyribose heterocycle, e.g., a morpholine structure (a non-natural, or modified, nucleoside or other structures described elsewhere herein). Thus, a series of such modified, non-natural, nucleosides linked together via an internucleoside backbone can also be considered to be an oligonucleotide (a non-natural, or modified, oligonucleotide). Further, the term “sugar” where used herein in the context of a nucleoside, is intended to include “non-sugar” heterocyclic compounds, such as morpholines, as the portion of the internucleoside backbone which is linked to the nucleobase.

Oligonucleotides useful in the compounds and methods disclosed herein also include those comprising entirely or partially of naturally occurring nucleobases. Naturally occurring nucleobases as defined herein, include adenine, guanine, thymine, cytosine, and uracil. Although 5-methylcytosine (5-me-C) is technically a naturally occurring nucleobase, for the purposes of the present disclosure it will be included in the list of non-natural (a.k.a., modified) nucleobases.

As noted above, oligonucleotides of the present disclosure may further include those comprised entirely or partially of modified nucleobases and their corresponding nucleosides. These modified nucleobases include, but are not limited to, 5-uracil (pseudouridine), dihydrouracil, inosine, ribothymine, 5-me-C, 7-methylguanine, hypoxanthine, xanthine, 5-hydroxymethyl cytosine, 2-aminoadenine, 2-methyladenine, 6-methyladenine, 2-propyladenine, N6-adenine, N6-isopentenyladenine, 2-methylthio-N6-isopentenyladenine, 2-methylguanine, 6-methylguanine, 2-propylguanine, 1-methylguanine, 7-methylguanine, 2,2-dimethylguanine, 2-thiouracil, 2-thiothymine, 2-thiocytosine, 5-fluorouracil, 5-bromouracil, 5-chlorouracil, 5-iodouracil, dihydrouracil, 5-methyl-2-thiouracil, 2-thiouracil, 4-thiouracil, 5-methyluracil, uracil-5-oxyacetic acid methylester, uracil-5-oxyacetic acid, 5-carboxymethylaminomethyl-2-thiouracil, hypoxanthine, xanthine, 4-acetylcytosine, 5-(carboxyhydroxylmethyl) uracil, 5-methoxycarboxymethyluracil, 5-methoxyuracil, 5-methyl-2-thiouracil, 3-(3-amino-3-N-2-carboxypropyl) uracil, 5-carboxymethylaminomethyluracil, 5-methylaminomethyluracil, 5-methoxyaminomethyl-2-thiouracil, alkynyl derivatives of pyrimidine bases including 5-propynyl uracil, and 5-propynyl cytosine, 6-azo uracil, 6-azo cytosine, 6-azo thymine, 4-thiouracil, 8-halo-adenines, 8-amino adenine, 8-thiol adenine, 8-thioalkyl adenine, 8-hydroxyl adenine, 5-trifluoromethyl uracil, 3-methylcytosine, 5-methylcytosine, 5-trifluoromethyl cytosine, 7-methylguanine, 7-methyladenine, 2-F-adenine, 2-amino-adenine, 8-azaguanine and 8-azaadenine, 7-deazaguanine, 8-halo-guanines, 8-amino guanine, 8-thiol guanine, 8-thioalkyl guanine, 8-hydroxyl guanine, 7-deazaadenine, 3-deazaguanine, 3-deazaadenine, beta-D-galactosylqueosine, beta-D-mannosylqueosine, 1-methylinosine, 2,6-diaminopurine, queosine, tricyclic pyrimidines, phenoxazine cytidine(1H-pyrimido[5,4-b][1,4]benzoxazin-2(3H)-one), and phenothiazine cytidine (1H-pyrimido[5,4-b][1,4]benzothiazin-2(3H)-one.

The present disclosure also encompasses oligonucleotides which comprise targeting sequences (base sequences) that are complementary to particular nucleic acid target sequences taught herein. A nucleic acid is a “complement” or is “complementary” to another nucleic acid when it is capable of base-pairing with the other nucleic acid according to the standard Watson-Crick, Hoogsteen or reverse Hoogsteen binding complementarity rules. Polynucleotides (nucleic acids) are described as “complementary” to one another when hybridization occurs in an antiparallel configuration between two single-stranded polynucleotides.

More particularly, “complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target, and as such, as is understood in the art, the targeting sequence of an antisense oligonucleotide of the present disclosure need not be 100% complementary to that of its target sequence to be specifically hybridizable. An oligonucleotide is specifically hybridizable when binding of the oligonucleotide to the target sequence of the DNA or RNA molecule interferes with the normal function of the target DNA or RNA to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, and in the case of in vitro assays, under conditions in which the assays are performed. An oligonucleotide and a target sequence are thus complementary to each other when a sufficient number of nucleobases of the oligonucleotide can hydrogen bond with the corresponding nucleobases of the target sequence, such that a desired effect will occur (e.g., antisense inhibition of a target nucleic acid, such as an AR coregulator).

For example, an oligonucleotide in which 18 of 20 nucleobases of the oligonucleotide are complementary to a target sequence, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligonucleotide which is 18 nucleobases in length having three noncomplementary nucleobases which are flanked by two regions of complete complementarity with the target nucleic acid, or are distributed in non-contiguous positions, would have 83% overall complementarity with the target sequence.

In other embodiments, the seed sequence of the antisense oligonucleotide provided herein is fully complementary (i.e. 100% complementary) to a target sequence of a nucleic acid, e.g., of an AR coregulator. As used herein, “fully complementary” means each nucleobase of the referenced portion of an oligonucleotide (e.g., the seed sequence) is capable of precise base pairing with the corresponding nucleobases of a target nucleic acid.

The term “target sequence” where used herein refers to a contiguous series of nucleobases in a specific nucleotide sequence (target region), for example of an mRNA. The term “target sequence” refers to a sequence that is a subsequence (portion or segment) of the target region, or to the entire sequence of the target region. A target sequence may include the 5′ terminal nucleobase of a nucleic acid sequence plus adjacent internal nucleobases of the sequence, or the 3′ terminal nucleobase plus adjacent internal nucleobases of the sequence, or only internal nucleobases within the sequence, or the target sequence may be 100% identical to the target region. In certain embodiments, a nucleic acid compound of the present disclosure comprises an oligonucleotide having a nucleobase sequence that, when written in the 5′ to 3′ direction, comprises the reverse complement of a target sequence of a nucleic acid target region to which it is targeted.

The terms “complementary” and “antisense” can be used interchangeably. Complementary also refers to polynucleotide sequences that are substantially complementary (antisense) over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches.

In certain embodiments, oligonucleotides of the present disclosure are synthesized using one or more modified nucleotides. As used herein, the terms “modified” and “modification” when used in the context of the constituents of a nucleotide monomer, i.e., sugar, nucleobase and internucleoside linkage (backbone), refer to non-natural changes to the chemical structure of these naturally occurring constituents or the substitutions of these constituents with non-naturally occurring ones, i.e., mimetics. For example, the “unmodified” or “naturally occurring” sugar ribose (of RNA) can be modified by replacing the hydrogen at the 2′-position of ribose with a methyl group. Similarly, the naturally occurring internucleoside linkage of nucleic acids is a 3′ to 5′ phosphodiester linkage that can be modified, in one embodiment, by replacing one of the non-bridging oxygen atoms of the phosphate linker with a sulfur atom to create a phosphorothioate linkage. Modified oligonucleotides are structurally distinguishable, but functionally interchangeable with naturally occurring or synthetic unmodified oligonucleotides and usually have enhanced properties such as increased resistance to degradation by exonucleases and endonucleases, or increased binding affinity.

As noted above, in certain embodiments, modifications to the oligonucleotides of the present disclosure encompass substitutions or changes in internucleoside linkages, sugar moieties, or nucleobases. Where used herein in reference to an oligonucleotide, the term “non-natural” or “unnatural” refers to an oligonucleotide which comprises at least one modification in an internucleoside linkage, a sugar, and/or a nucleobase thereof, wherein such modified internucleoside linkage, modified sugar, and/or modified nucleobase is not found naturally in DNA or RNA (unless specifically defined otherwise herein)

Non-naturally occurring internucleoside linkages of the oligonucleotides of the present disclosure include those that contain a phosphorus atom and also those that do not contain a phosphorus atom. Numerous phosphorus-containing modified oligonucleotide backbones are known in the art and may be used in the oligonucleotides of the present disclosure. Examples of phosphorus-containing internucleoside linkages of non-natural (modified) oligonucleotide backbones which may occur in the presently disclosed oligonucleotides include, but are not limited to, phosphorothioate, phosphorodithioate, phosphoramidite, phosphorodiamidate, morpholino, phosphotriester, aminoalkylphosphotriester, phosphonate, chiral phosphorothioates, methyl and other alkyl phosphonates including 3′-alkylene phosphonate, 5′-alkylene phosphonate and chiral phosphonate, phosphinate, phosphoramidates including 3′-amino phosphoramidate and aminoalkylphosphoramidate, thionophosphoramidate, thionoalkylphosphonate, thionoalkylphosphotriester, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage, and oligonucleotides having inverted polarity comprise a single 3′ to 3′ linkage at the 3′-most internucleotide linkage i.e., a single inverted nucleoside residue which may be abasic (the nucleobase is missing or has a hydroxyl group in place thereof) linkages. Examples of U.S. patents that teach the preparation of such phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

As noted above, in some embodiments, the internucleoside linkages are without phosphorus atoms and may instead comprise short chain alkyl or cycloalkyl internucleoside linkages, mixed heteroatom and alkyl or cycloalkyl internucleoside linkages, or one or more short chain heteroatomic or heterocyclic internucleoside linkages. In further embodiments, the non-naturally occurring internucleoside linkages are uncharged and in others, the linkages are achiral. In some embodiments, the non-naturally occurring internucleoside linkages are uncharged and achiral, such as peptide nucleic acids (PNAs).

It is understood that the sequence set forth in each sequence or SEQ ID NO contained herein is independent of any modification to sugar moieties, internucleoside linkages, or nucleobases of the sequence, unless otherwise specified. As such, antisense oligonucleotides of the present disclosure may be defined by a complementary correspondence to a sequence or SEQ ID NO disclosed herein, or segment thereof, and may comprise, independently, one or more modifications to a sugar moiety, an internucleoside linkage, or a nucleobase. Other embodiments of oligonucleotide backbones include siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Examples of U.S. patents that teach the preparation of such non-phosphorus containing oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In certain oligonucleotide mimetics of the present disclosure, both the sugar moiety and the internucleoside linkage, i.e., the backbone, of the nucleotide units are replaced with non-natural groups. One such oligomeric compound is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Examples of U.S. patents that teach the preparation of PNA compounds include, but are not limited to, U.S. Pat. Nos. 5,539,082; 5,714,331; and 5,719,262.

The oligonucleotides described herein stabilized against nucleolytic degradation such as by the incorporation of a modification, e.g., a nucleotide modification. The oligonucleotides can include a non-natural nucleoside linkage such as a phosphorothioate linkage as the first, second, and/or third internucleotide linkage at the 5′ or 3′ end of the oligonucleotide sequence. In certain embodiments, the oligonucleotides can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) nucleotide. In a particular embodiment, the oligonucleotides include at least one 2′-O-methyl-modified nucleotide, and in some embodiments, all of the nucleotides include a 2′-O-methyl modification.

As noted elsewhere herein, the oligonucleotide can be further modified so as to be conjugated to an organic moiety such as a biogenic molecule that is selected to improve stability, distribution and/or cellular uptake of the oligonucleotide, e.g., cholesterol, forming the nucleic acid compound of the present disclosure. Such an organic moiety can be attached, e.g., to the 3′ or 5′ end of the oligonucleotide, and/or at the 2′ position of the sugar moiety of a nucleotide of the oligonucleotide, such as the 2′ ribose position.

The nucleic acid compound can further be in isolated form or can be part of a pharmaceutical composition, such as a pharmaceutical composition formulated for parental administration. The pharmaceutical compositions can contain one or more nucleic acid compounds, and in some embodiments will contain two or more inhibitory nucleic acid compounds, each one directed to a different target gene.

The oligonucleotides can be delivered in any of a variety of forms, including in liposomes as described above, and via expression vectors. The oligonucleotide can be endogenously expressed from transcription units inserted into DNA or RNA vectors. The recombinant vectors can be DNA plasmids or viral vectors for example. Viral vectors suitable for producing the presently disclosed oligonucleotides capable of reducing expression or activity of an AR coregulator can be constructed based on, but not limited to, adeno-associated virus, retrovirus, lentivirus, adenovirus, or alphavirus. The recombinant vectors which contain a nucleic acid for expressing the oligonucleotides disclosed herein can be delivered as described above and can persist in target cells. Alternatively, viral vectors can be used that provide for transient expression of the oligonucleotides. Such vectors can be repeatedly administered as necessary. Once expressed, in one embodiment, the oligonucleotides may interact with the target RNA and inhibit mRNA activity for example. The delivery vehicles (vectors) for the oligonucleotides optionally comprise an expression construct which includes an enhancer sequence, a promoter sequence, and other sequences necessary for expression of the products of the oligonucleotide sequence desired to be produced. In one embodiment, the promoter is cell-specific. The term “cell-specific” means that the particular promoter selected for the recombinant vector can direct expression of the selected transgene only in a particular cell type. As one example, the promoter is specific for expression in prostate cells. A number of viruses can be used in connection with the methods described herein, including papovaviruses, e.g., SV40, adenovirus, vaccinia virus, adeno-associated virus, herpesviruses including HSV and EBV, and retroviruses of avian, murine, and human origin. In certain embodiments, lentiviral vectors can be used in connection with the methods described herein. In certain embodiments, the lentiviral vector can be a doxycycline-inducible lentiviral vector engineered to express one or more shRNAs or siRNAs.

Specific vectors which may be used include, but are not limited to, adeno-associated virus vectors (e.g., as disclosed in U.S. Pat. Nos. 5,139,941, 5,436,146, and 5,622,856), an attenuated or gutless adenoviral vectors, (e.g., as disclosed in U.S. Pat. No. 5,935,935), lentiviral vectors (such as are disclosed in U.S. Pat. Nos. 5,665,577; 5,994,136; and 6,013,516), plasmids or synthetic (non-viral) vectors (such as disclosed in U.S. Pat. Nos. 4,394,448 and 5,676,954), and/or nanoparticles (such as disclosed, for example, in U.S. Pat. Nos. 6,217,912; 7,514,098; and 8,323,618), retroviral vectors (such as are disclosed in U.S. Pat. Nos. 5,672,510; 5,707,865; and 5,817,491), herpes virus vectors (such as are disclosed in U.S. Pat. No. 5,288,641), and sindbis virus vectors and papilloma virus vectors (such as are disclosed in EP 820 773). The vectors may be either monocistronic, bicistronic, or multicistronic. A recombinant vector (e.g., lenti-, parvo-, AAV) sequence can be packaged as a “particle” for subsequent infection (transduction) of a cell, ex vivo, in vitro or in vivo. Where a recombinant vector sequence is encapsulated or packaged into an AAV particle, the particle can also be referred to as a “rAAV.” Such particles include proteins that encapsulate or package the vector genome. Particular examples include viral envelope proteins, and in the case of AAV, capsid proteins.

Thus, the oligonucleotides of the present disclosure may be used as a form of gene therapy. The term “gene therapy” as used herein means genetic modification of cells by the introduction of exogenous DNA or RNA into these cells, such as via an expression vector containing the oligonucleotide, for the purpose of expressing or replicating one or more peptides, polypeptides, proteins, oligonucleotides, or polynucleotides in vivo for the treatment or prevention of disease or deficiencies in humans or animals. Examples of gene therapy are disclosed for example in U.S. Pat. No. 5,399,346. Any suitable route of administration of the oligonucleotide-containing vector may be employed. For example, parenteral (subcutaneous, subretinal, suprachoroidal, intramuscular, intravenous, transdermal) and like forms of administration may be employed. Dosage formulations include injections, implants, or other known and effective gene therapy delivery methods.

Delivery of the oligonucleotide-expressing vectors can be systemic, such as by intravenous or intra-muscular administration, direct administration to a tumor site, such as a prostate tumor, by administration to target cells ex-planted from a subject followed by reintroduction into the subject, or by any other means that would allow for introduction into the desired target cell. The therapeutic and/or pharmaceutical compositions, in non-limiting embodiments, contain viral particles per dose in a range of, for example, from about 10⁴ to about 10¹¹ particles, from about 10⁵ to about 10¹⁰ particles, or from about 10⁶ to about 10⁹ particles. In the context of AAV vectors, vector genomes are provided in in a range of, for example, from about 10⁴ to about 10¹⁴ vector genomes, from about 10⁵ to about 10¹³ vector genomes, from about 10⁶ to about 10¹³ vector genomes, from about 10⁷ to about 10¹³ vector genomes, from about 10⁸ to about 10¹³ vector genomes, or from about 10⁹ to about 10¹³ vector genomes. Such doses/quantities of AAV vector are useful in the methods set forth herein.

Because nucleases that cleave the phosphodiester linkages are expressed in almost every cell, unmodified nucleic acid molecules such as the inhibitory oligonucleotides of the present disclosure may be modified to resist degradation, as described above for example. Other biogenic molecules may be conjugated to the oligonucleotides to improve their ability to resist degradation, target certain cells, or to cross barriers like cell membranes or the blood brain barrier. Examples of biogenic molecules that can be conjugated to the oligonucleotides include lipids such as, but not limited to, stearic acid, palmitic acid, docosanoic acid, docosahexanoic acid, docosahexaenoic acid, cholesterol, tocopherol, and other C12-C22 saturated or unsaturated fatty acids; peptides such as but not limited to, cell-penetrating peptides (CPPs) such as penetratin, HIV-1 Tat peptides, pVEC-Cadherin 615-634, polyarginines (6-12), and transportan, linear and cyclic RGD-containing peptides, and SPACE peptide; receptor-specific ligands; aptamers (synthetic oligoribonucleotides); antibodies or antibody fragments; CpG-containing oligonucleotides; polyamines, such as spermine and spermidine; polymers such as dendrimers and polyethylene glycols (e.g., PEG 0.6 kDa-5,000 kDa); and saccharides such as N-acetylgalactosamine (GalNAc) and cyclodextrins. The biogenic molecule may be conjugated to the oligonucleotide by any suitable means, such as via linker or a cleavable bond such as but not limited to disulfide, thioether, pH sensitive (e.g., hydrazone or carboxymethylmaleic anhydride), or ethylene glycol.

The oligonucleotides or nucleic acid compounds of the present disclosure may be delivered in the form of nanoparticles and microparticles which encapsulate the nucleic acid compounds within liposomes of cationic lipids or within PEG, for example. These delivery systems can enhance intracellular delivery either by protecting the nucleic acid compound from nuclease degradation and/or by promoting absorptive endocytosis. Further, the addition of dioleylphosphatidylethanolamine to liposome delivery systems results in the destabilization of endosomal membranes and promotion of release of the oligonucleotide after endocytosis. The nucleic acid compounds can be administered to cells by a variety of other methods known to those of skill in the art, including, but not limited to, ionophoresis, or by incorporation into other vehicles, such as hydrogels, cyclodextrins, biodegradable nanocapsules, and bioadhesive microspheres, or by proteinaceous vectors. In one example, the nucleic acid compounds can be delivered via the nanoparticle system shown in U.S. Patent Application Publication 2019/0255088. The liposomes may comprise amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323.

In certain embodiments, the nanoparticles which contain the nucleic acid compounds of the present disclosure may comprise a pharmaceutically acceptable carrier such as, but not limited to, poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid, polycaprolactone, polyvalerolactone, poly (anhydrides), copolymers of polycaprolactone with polyethylene glycol, copolymers of polylactic acid with polyethylene glycol, polyethylene glycol; fibrin, Gelfoam™ (which is a water-insoluble, off-white, nonelastic, porous, pliable gel foam prepared from purified gelatin and water for injection), and combinations and blends thereof. Copolymers can comprise from about 1% to about 99% by weight of a first monomer unit such as ethylene oxide and from 99% to about 1% by weight of a second monomer unit such as propylene oxide. Blends of a first polymer such as gelatin and a second polymer such as poly-L-lactic acid or polyglycolic acid can comprise from about 1% to about 99% by weight of the first polymer and from about 99% to about 1% of the second polymer.

The oligonucleotides or nucleic acid compounds can be delivered directly by systemic administration such as using oral formulations or stereotactic injection into prostate or prostate tumor, typically in saline with chemical modifications to enable uptake, or other methods described elsewhere herein. In certain embodiments, such as when the oligonucleotide of the nucleic acid compound has a phosphorothioate backbone, the oligonucleotide binds to serum proteins, slowing excretion by the kidney. The aromatic nucleobases also interact with other hydrophobic molecules in serum and on cell surfaces. In certain embodiments, siRNA delivery systems involve complexing the RNA with cationic and neutral lipids, although encouraging results have also been obtained using peptide transduction domains and cationic polymers. Including PEGylated lipids in the formulation prolongs the circulating half-life of the particles.

As noted, one type of optimization of single-stranded DNA or RNA oligonucleotides is the use of chemical modifications to increase the nuclease resistance such as the introduction of phosphorothioate (“PS”) linkages in place of the phosphodiester bond. This modification improves protection from digestion by nucleases. PS linkages also improved binding to serum proteins in vivo, increasing half-life and permitting greater delivery of active compound to tissues. Chemical modifications to subunits of the nucleotides can also improve potency and selectivity by increasing binding affinity of oligonucleotides for their complementary sequences. Examples of such modifications to the nucleoside sugars include 2′-O-methyl (2′-O-Me), 2′-fluoro (2′-F), and 2′-O-methoxyethyl (2′-MOE) RNA, and others as discussed elsewhere herein. Even more affinity can be gained using oligonucleotides modified with locked nucleic acid (LNA), which contains a methylene bridge between the 2′ and 4′ position of the ribose. This bridge “locks” the ribose ring in a conformation that is ideal for binding, leading to high affinity for complementary sequences. Related bridged nucleic acid (BNA) compounds have been developed and share these favorable properties. Their high affinity has permitted the development of far shorter oligonucleotides than previously thought possible which nonetheless retain high potency. The chemistry for introducing 2′-O-Me, 2′-MOE, 2′-F, or LNA into oligonucleotides is compatible with DNA or RNA synthesis, allowing chimeras with DNA or RNA bases to be easily obtained. This compatibility allows the properties of chemically modified oligonucleotides to be fine-tuned for specific applications, which is a major advantage for development that makes LNAs and other BNAs convenient tools for many applications.

Therapeutic administration of the active agents described herein include any method by which a nucleic acid (e.g., DNA or RNA), as known to one of ordinary skill in the art. For treatment of aggressive prostate cancer, delivery may be via, for example, oral administration and/or injection into the prostate gland or tumor or both.

In certain embodiments, the active agents can be delivered to an organelle, a cell, a tissue, a tumor or an organism via one or more injections (i.e., a needle injection), such as, for example, orally, subcutaneously, intradermally, intramuscularly, intravenously, or intraperitoneally.

A described inhibitory nucleic acids or other active agent can be incorporated into pharmaceutical compositions suitable for administration. For example, pharmaceutical compositions can comprise one or more the active agents and a pharmaceutically acceptable carrier.

Then active agent may be provided in a sustained release composition. The use of immediate or sustained release compositions depends on the nature of the condition being treated. If the condition consists of an acute or over-acute disorder, treatment with an immediate release form can be conducted over a prolonged release composition. Alternatively, for certain preventative or long-term treatments, a sustained release composition may be appropriate.

The active agent can be administered in a single dose or in multiple doses. Where the administration of the active agent is by infusion, the infusion can be a single sustained dose or can be delivered by multiple infusions. Injection of the active agent can be directly into the tissue at or near the site of aberrant or unwanted target gene expression. Multiple injections of the active agent can be made into the tissue, for example, into the prostate gland, into the prostate tumor, or near the tumor.

In addition to treating pre-existing aggressive or non-aggressive cancers, active agents of the disclosure can be administered prophylactically in order to prevent or slow the conversion of a non-aggressive prostate cancer to an aggressive form. The active agent can be employed in combination therapies, meaning that the present compositions can be administered concurrently with, prior to, or subsequent to, one or more other desired therapeutic agents or medical procedures. The combination of therapies (therapeutic agents or procedures) to employ in a combination regimen will take into account compatibility of the desired therapeutic agents and/or procedures and the desired therapeutic effect to be achieved. It will also be appreciated that the therapies employed can achieve a desired effect for the same disorder (for example, a compound described herein can be administered concurrently with another therapeutic agent used to treat the same disorder), or they can achieve different effects (e.g., control of any adverse effects).

Turning now to particular embodiments of the present disclosure, results are provided herein by utilizing a pharmaceutical and molecular biology-based approach to inhibit ovarian cancer growth and prevent metastases in the platinum-based chemotherapy-resistant setting showing the efficacy of the cell lines, model system, and the combined targeting approach for the treatment of resistant and recurrent ovarian cancer. There are multiple applications for this technology including the prevention of platinum-therapy cell escape, studying the etiology of platinum-chemotherapy resistance, and development of new combination therapies to overcome, prevent or combat cisplatin-based chemotherapeutic resistance in HGSOC in a physiologically relevant model system.

OVCAR-8 and the cisplatin-resistance (CPR) derivative OVCAR-8 CPR are cell lines obtained from a patient and represent human high-grade serous carcinoma. We transduced these cells with lentivirus carrying an expression vector containing the firefly luciferase gene under the control of a strong promoter. We performed two rounds of lentiviral transduction, followed by selection for puromycin resistance at 2 μg/mL concentration. We confirmed the firefly luciferase gene's expression by adding the D-luciferin substrate to cell protein lysates. The average luminescence detected in the luciferase transduced cells and non-transduced cells is shown in Table 1.

TABLE 1
Average luminescence signal detected in cell lysates.

| Samples | Replicate 1 | Replicate 2 | Replicate 3 |
|---|---|---|---|
| Passive lysis buffer | 2 | 5 | 2 |
| OVCAR-8 parental | 4 | 3 | 4 |
| OVCAR-8 luc | 332 | 294 | 353 |
| OVCAR-8 CPR parental | 4 | 3 | 4 |
| OVCAR-8 CPR luc | 130 | 143 | 138 |

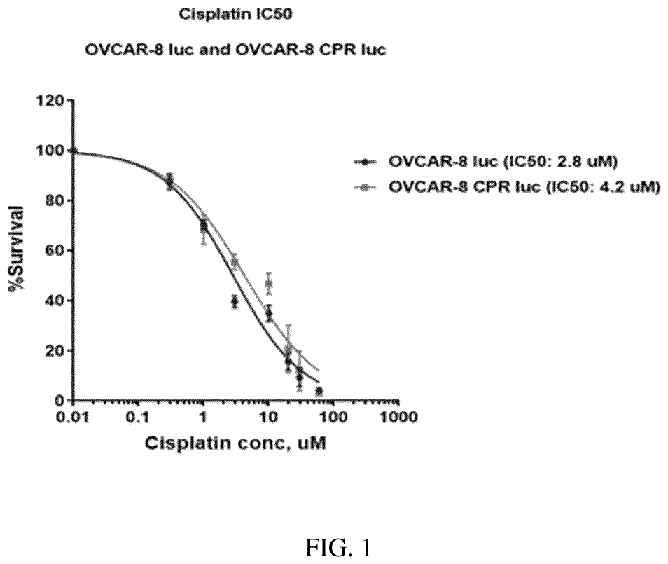

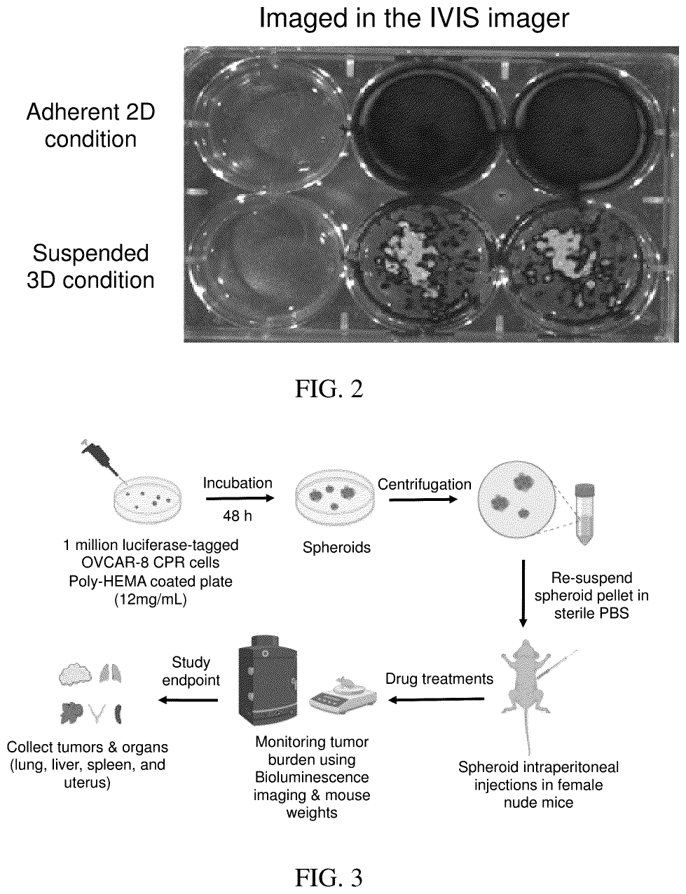

Next, we confirmed that these cells retained their inherent response to cisplatin. As shown in FIG. 1, we performed a cisplatin-dose response curve and calculated the 50% inhibitory concentration value (IC50) for the OVCAR-8 Luc and OVCAR-8 CPR Luc cells and found that the IC50 of the OVCAR-8 CPR Luc cells to cisplatin was twice that of the cisplatin-sensitive cells (OVCAR-8 Luc). To determine if luciferase activity could be visualized in vitro we plated the OVCAR-8 CPR Luc cells in 2-dimensional (2D) adherent and suspended three-dimensional (3D) spheroid conditions. As shown in FIG. 2, the cells produced high levels of luminescent signal.

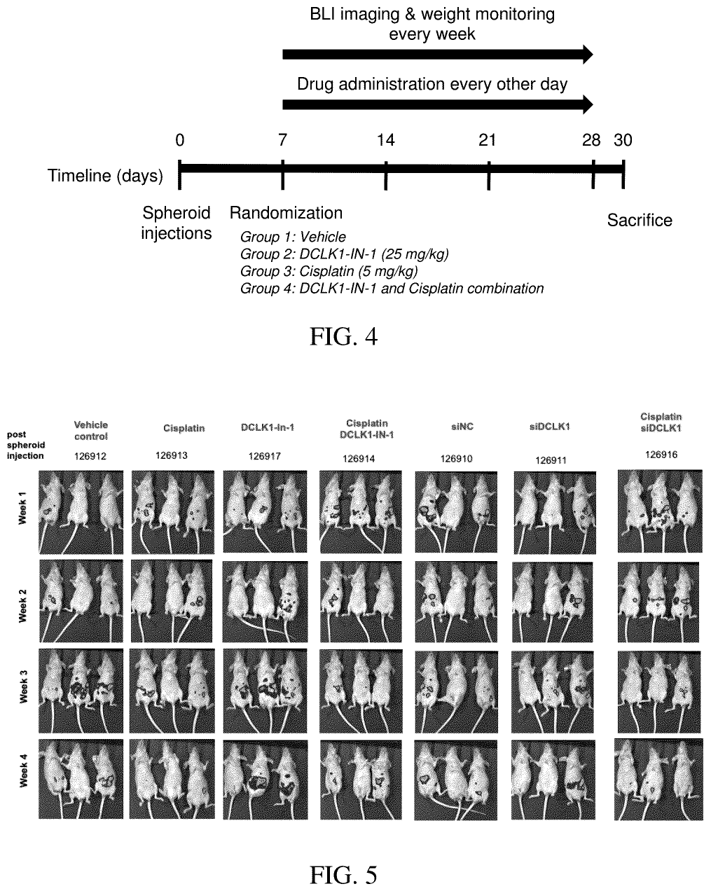

Ovarian cancer progression occurs as single cells or multi-cellular spheroids disseminated from the primary tumor site, accumulate in ascites, and adhere to the peritoneal cavity to generate metastases. It is well known that chemotherapy alters the behavior of tumor cells in many ways, including the activation of plasticity pathways involving the epithelial to mesenchymal transition (EMT) and the enrichment of cells with increased tumor-initiating capacity, known as “cancer stem cells” (CSCs)^{3,4} that can promote disease recurrence.^{3,5-8} The doublecortin-like kinase 1 (DCLK1) is a putative CSC marker and promoter as a chemoresistance driver in HGSOC. DCLK1 expression is elevated in ovarian cancer tissues and associated with poor progression-free and overall survival. We have also observed an increase of DCLK1 in cisplatin-resistance HGSOC cells, relative to cisplatin-sensitive cells (data not shown). To evaluate whether pharmacologic treatment with an inhibitor of DCLK1 or self-assembled siRNAs (SAMiRNAS) targeted to DCLK1 could overcome resistance to cisplatin, we generated a bioluminescent in vivo model of peritoneal cisplatin-resistant HGSOC using the luciferase-labeled OVCAR-8 CPR Luc cells. As shown in FIG. 3, 1×10⁶ OVCAR-8 CPR Luc cells grown as spheroids on poly-HEMA coated plates were injected intraperitoneally into female nude mice. We then evaluated whether treatment with a pharmacologic inhibitor of DCLK1 alone or in combination with cisplatin could reduce tumor growth and peritoneal metastases, see FIG. 4 for procedures.

One week after the injection of OVCAR-8 CPR Luc spheroids, we initiated the whole animal in vivo imaging to monitor tumor growth in real time. As shown in FIG. 5, we observed a strong luciferase signal at week 1. The luciferase signal also decreased in the single-agent and double-agent treatment groups over the next 3 weeks relative to the control group.

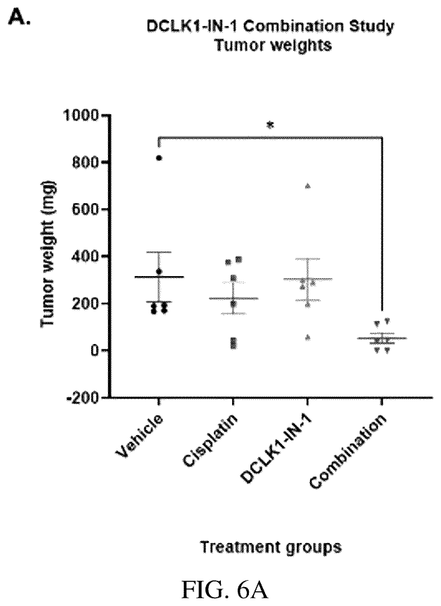

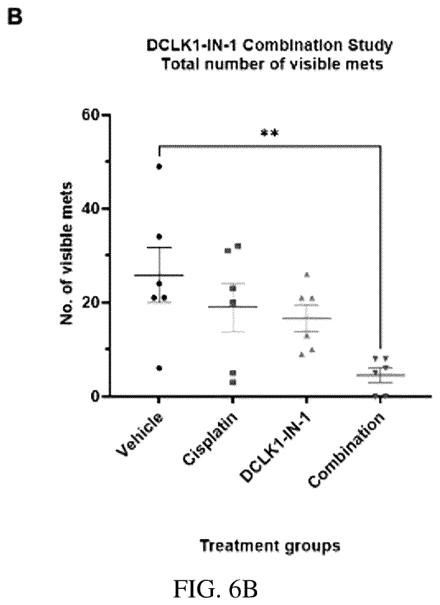

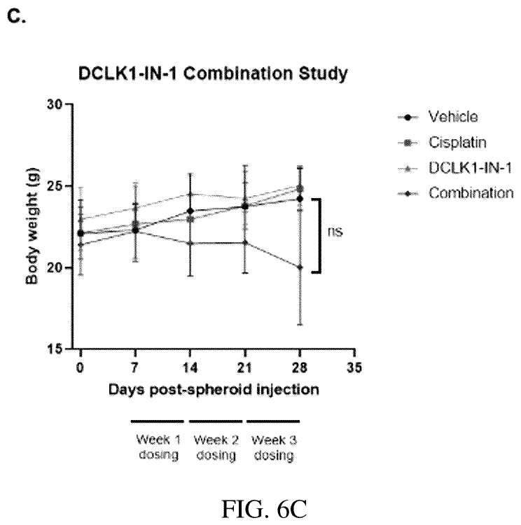

For the DCLK1-IN-1 combination study, we observed a significant decrease in tumor weights and the number of metastases for the DCLK1-IN-1+Cisplatin treatment combination groups relative to vehicle only control group (FIG. 6A-6B). We did not observe any significant differences in the mouse body weight among the groups (FIG. 6C).

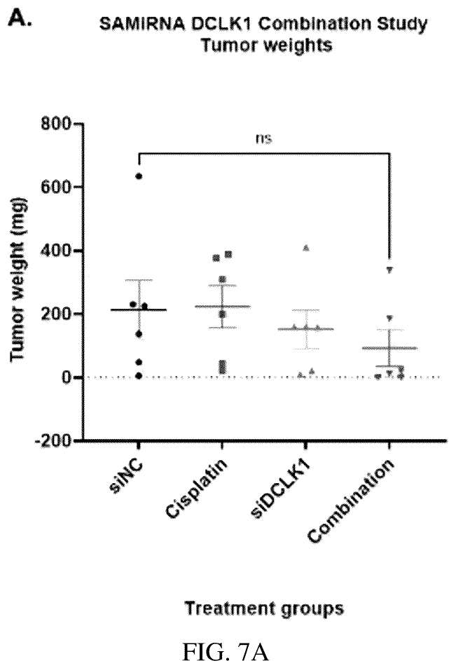

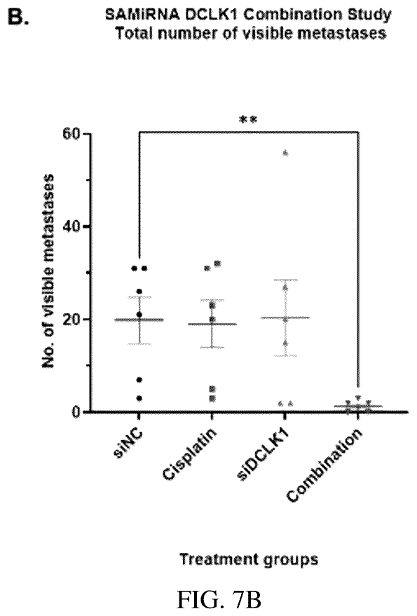

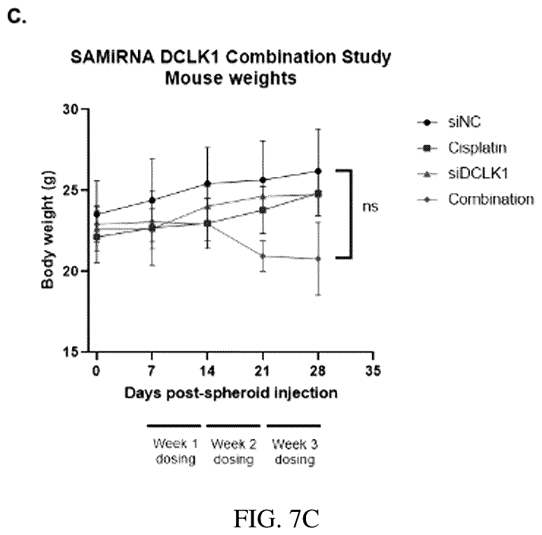

Similarly, for the SAMiRNA DCLK1 targeting combination study, we observed a decrease in tumor weights for the siDCLK1 and combination treatment groups, however, it did not reach significance. We did observe a significant decrease in the number of metastases for the siDCLK1 and cisplatin treatment combination groups relative to the siNC control group (FIG. 7A-7B). We did not observe any significant differences in the mouse body weight among the groups (FIG. 7C).

In summary, disclosed herein is a pharmaceutical and molecular biology-based approach to inhibit ovarian cancer growth and prevent metastases in the platinum-based chemotherapy-resistant setting showing the efficacy of the cell lines, model system, and the combined targeting approach for the treatment of resistant and recurrent ovarian cancer. There are multiple applications for this technology including the prevention of platinum-therapy cell escape, studying the etiology of platinum-chemotherapy resistance, and development of new combination therapies to overcome, prevent or combat cisplatin-based chemotherapeutic resistance in HGSOC in a physiologically relevant model system. The cell lines and model system are not limited to the use of transduced OVCAR-8 cell lines, but can be made with any appropriate HGSOC cell line that can be effectively transduced with the firefly luciferase gene, or any other gene which causes bioluminescense in the xenograft tumors in vivo.

In at least one embodiment, the present disclosure is directed to a method of screening a chemical substance as a drug candidate against High-grade serous ovarian carcinoma (HGSOC), including the steps of comprising (a) providing mice having xenograft tumors caused by at least one of (1) a cell line comprising HGSOC cells which have been transduced with firefly luciferase gene (Luc) to form HGSOC-Luc cells, and (2) a cell line comprising cisplatin-resistant HGSOC (HGSOC-CPR) cells which have been transduced with Luc to form HGSOC-CPR Luc cells, (b) administering the chemical substance to the mice for a predetermined treatment period, and (c) measuring an effect that the chemical substance has on the mice after the predetermined treatment period, wherein the effect is a significant decrease in at least one of (i) the weight of the xenograft tumors and (ii) the number of HGSOC metastases in the mice, wherein the significant decrease identifies the chemical substance as a candidate drug capable of ameliorating HGSOC in a subject in need of such treatment.

In another embodiment, the present disclosure is directed to a method of screening a chemical substance as a drug candidate against High-grade serous ovarian carcinomas (HGSOC), including the steps of (a) providing mice having xenograft tumors caused by injection of at least one of (1) a cell line comprising OVCAR-8 cells which have been transduced with firefly luciferase gene (Luc) to form OVCAR-8-Luc cells and (2) a cell line comprising cisplatin-resistant OVCAR-8 (OVCAR-8-CPR) cells which have been transduced with Luc to form OVCAR-8-CPR Luc cells, (b) administering the chemical substance to the mice for a predetermined treatment period, and (c) measuring an effect that the chemical substance has on the mice after the predetermined treatment period, wherein the effect is a significant decrease in at least one of (i) the weight of the xenograft tumors and (ii) the number of HGSOC metastases in the mice, wherein the significant decrease identifies the chemical substance as a candidate drug capable of ameliorating HGSOC in a subject in need of such treatment.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus, the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
1. Coleman, R. L., Monk, B. J., Sood, A. K. & Herzog, T. J. Latest research and treatment of advanced-stage epithelial ovarian cancer. Nat Rev Clin Oncol 10, 211-224 (2013). https://doi.org:10.1038/nrclinonc.2013.5
2. Herzog, T. J. & Pothuri, B. Ovarian cancer: a focus on management of recurrent disease. Nat Clin Pract Oncol 3, 604-611 (2006). https://doi.org:10.1038/ncponc0637
3. Ahmed, N., Abubaker, K., Findlay, J. & Quinn, M. Cancerous ovarian stem cells: obscure targets for therapy but relevant to chemoresistance. J Cell Biochem 114, 21-34 (2013). https://doi.org:10.1002/jcb.24317
4. Lengyel, E. Ovarian cancer development and metastasis. Am J Pathol 177, 1053-1064 (2010). https://doi.org:10.2353/ajpath.2010.100105
5. Abubaker, K. et al. Short-term single treatment of chemotherapy results in the enrichment of ovarian cancer stem cell-like cells leading to an increased tumor burden. Mol Cancer 12, 24 (2013). https://doi.org:10.1186/1476-4598-12-24
6. Ricci, F. et al. Patient-derived ovarian cancer xenografts re-growing after a cisplatinum treatment are less responsive to a second drug re-challenge: a new experimental setting to study response to therapy. Oncotarget 8, 7441-7451 (2017). https://doi.org:10.18632/oncotarget.7465
7. Alvero, A. B. et al. Molecular phenotyping of human ovarian cancer stem cells unravels the mechanisms for repair and chemoresistance. Cell Cycle 8, 158-166 (2009). https://doi.org:10.4161/cc.8.1.7533
8. Latifi, A. et al. Cisplatin treatment of primary and metastatic epithelial ovarian carcinomas generates residual cells with mesenchymal stem cell-like profile. J Cell Biochem 112, 2850-2864 (2011). https://doi.org:10.1002/jcb.23199

What is claimed is:

1. A method of screening a chemical substance as a drug candidate against High-grade serous ovarian carcinoma (HGSOC), comprising:
providing mice having xenograft tumors caused by at least one of (1) a cell line comprising HGSOC cells which have been transduced with firefly luciferase gene (Luc) to form HGSOC-Luc cells, and (2) a cell line comprising cisplatin-resistant HGSOC (HGSOC-CPR) cells which have been transduced with Luc to form HGSOC-CPR Luc cells;
administering the chemical substance to the mice for a predetermined treatment period; and
measuring an effect that the chemical substance has on the mice after the predetermined treatment period, wherein the effect is a significant decrease in at least one of (i) the weight of the xenograft tumors and (ii) the number of HGSOC metastases in the mice, wherein the significant decrease identifies the chemical substance as a candidate drug capable of ameliorating HGSOC in a subject in need of such treatment.

2. A method of screening a chemical substance as a drug candidate against High-grade serous ovarian carcinomas (HGSOC), comprising:
providing mice having xenograft tumors caused by injection of at least one of (1) a cell line comprising OVCAR-8 cells which have been transduced with firefly luciferase gene (Luc) to form OVCAR-8-Luc cells and (2) a cell line comprising cisplatin-resistant OVCAR-8 (OVCAR-8-CPR) cells which have been transduced with Luc to form OVCAR-8-CPR Luc cells;
administering the chemical substance to the mice for a predetermined treatment period; and
measuring an effect that the chemical substance has on the mice after the predetermined treatment period, wherein the effect is a significant decrease in at least one of (i) the weight of the xenograft tumors and (ii) the number of HGSOC metastases in the mice, wherein the significant decrease identifies the chemical substance as a candidate drug capable of ameliorating HGSOC in a subject in need of such treatment.


SEQUENCE LISTING (1 sequence)

SEQ ID NO: 1 - RNA, 19 nt, synthetic construct
  source 1..19: mol_type = other RNA
ggcgacttgcctgagcggg